(19) United States
(12) Patent Application Publication
(10) Pub. No.: US 2007/0224613 A1
(43) Pub. Date: Sep. 27, 2007

(54) Massively Multiplexed Sequencing

(76) Inventor: Michael Paul Strathmann, Seattle, WA (US)

Correspondence Address: MICHAEL STRATHMANN, 1205 8TH AVE W, SEATTLE, WA, 98119, US

(21) Appl. No.: 11/676,302

(22) Filed: Feb. 18, 2007

Related U.S. Application Data
(60) Provisional application No. 60/774,928, filed on Feb. 18, 2006.

(51) Int. Cl. C12Q 1/68

(52) U.S. Cl. 435/6; 977/924

(57) ABSTRACT

The present invention provides multiplexed methods for analyzing polynucleotides associated with sample tags. The multiplexed information is deconvoluted by single-molecule and more generally single-particle detection methods. In particular, a method for determining nucleic acid sequence information is provided.

### 1. RELATED APPLICATION DATA

This application claims the benefit of U.S. Provisional Application No. 60/774,928 filed on Feb. 18, 2006, which is incorporated herein by reference.

### 2. FIELD OF THE INVENTION

The present invention is related to the field of molecular biology, and provides multiplexed methods for analyzing nucleic acids, in particular nucleic acid sequencing.

### 3. BACKGROUND

The ability to rapidly and inexpensively sequence DNA will accelerate the development of pharmacogenomics, i.e. drugs and other medical treatments tailored to the genetic makeup of an individual. The significance of improvements to DNA sequencing methodologies is underscored by the stated goal of the National Human Genome Research Center to reduce the cost of sequencing a human genome to $1000.

Several methods for massively parallel sequencing are being commercialized (see for example, 454 Life Sciences, Solexa, Helicos and Agencourt) which rely on a sequencing-by-synthesis approach. This approach relies on a polymerase to incorporate one of the four bases per sequencing step in a replicating DNA strand (the template), followed by detection of the base. Typically, many identical DNA strands are sequenced simultaneously in order to produce enough signal for detection of the incorporated base. These replicating strands must remain “in sync” through each step of the sequencing process so that signals do not become jumbled. The result can be very short sequencing reads on the order of 20-25 bases. The process can be made highly parallel by performing the sequencing steps on different clusters of DNA strands in the same reaction vessel and recording signals simultaneously. Variations in the sequencing-by-synthesis approach have resulted in longer sequencing reads (e.g. 454 Life Sciences) but the tradeoff is increased overall cost.

Another strategy for massively parallel sequencing involves multiplexing many different templates that have been subjected to a standard sequencing reaction (for example, Sanger chain-termination reactions). The multiplexed sequencing reactions are separated by size, for example using a standard polyacrylamide gel, and the templates present in any fraction are identified by deconvolution of the multiplexed mixture. As with traditional Sanger sequencing, the sequence of a template is determined from the size-separated “ladder”. The key to these approaches is the method for multiplexing the templates and the method for deconvoluting the fractionated reaction products.

Van Ness describes the use of mass tags that can be detected by mass spectrometry (PCT Pat. Pub. No. WO 97/27331). Different tags are attached to the 5′ end of a sequencing primer. Each tagged primer is used to sequence a different template by the chain-termination method. The different reactions are pooled and fractionated by size (i.e. sequencing products are collected from the end of a capillary electrophoresis device). The tags present in each fraction are assayed by mass spectrometry. This information is deconvoluted to reproduce the “sequence ladders” of the different templates. The method is limited by the number of different tags that can be synthesized, which in turn limits the number of multiplexed templates. The method is limited since it is not parallel until the sequencing reactions are pooled.

Strathmann overcomes the limitations of Van Ness' method by employing nucleic acid tags instead of mass tags (U.S. Pat. No. 6,480,791). The number of different nucleic acid tags is enormous and simple to achieve, which permits very deep multiplexing. The deconvolution of fractionated sequencing reaction products is achieved by hybridization of nucleic acid tags to a DNA microarray comprising sequences that are complementary to the tags. The result is a massively parallel sequencing method capable of long read lengths. DNA microarrays are expensive, however and typically require 12 hours or longer to achieve good signal to noise ratios with complex samples. The time and expense to sequence a human genome may still be too prohibitive for “personal genomics”.

What is needed in the art is a method to sequence DNA very rapidly and inexpensively. The instant invention addresses this need by providing a massively-multiplexed sequencing method and novel deconvolution strategies that eliminate the need for microarrays.

### 4. BRIEF DESCRIPTION OF THE FIGURES

FIG. 1a is a drawing of a preferred embodiment of a sample tag joined to a sample polynucleotide.

FIG. 1b is a drawing of a preferred embodiment of sequencing primers and amplification primers for preparing and analyzing sequencing reaction products that are pooled prior to fractionation.

### 5. SUMMARY

It is an object of the invention to provide massively multiplexed methods for analyzing a collection of polynucleotides, particularly for generating nucleic acid sequence information. More specifically, the method employs Sanger or Maxam and Gilbert nucleic acid sequencing reactions carried out on a collection of sample polynucleotides cloned into sample-tagged vectors so that a sample tag preferably is joined to one sample polynucleotide. The sample tags are used to deconvolute the sequence information derived from the different sample polynucleotides. Deconvolution is achieved through single-molecule and more generally, single-particle detection methods.

### 6. DETAILED DESCRIPTION OF PREFERRED EMBODIMENTS

### 6.1 Definitions

A “sequence element” or “element” as used herein in reference to a polynucleotide is a number of contiguous bases or base pairs in the polynucleotide, up to and including the complete polynucleotide. When referring to a sequence element with a particular property, the sequence element consists of the bases or base pairs that contribute to the property or are defined by the property.

The term “sample” as used herein refers to a polynucleotide or that element of a polynucleotide which will be analyzed for some property according to the method of this invention. For example, a sample polynucleotide may be joined to other sequence elements to form a larger polynucleotide in order to practice the invention. The element of the larger polynucleotide that is homologous to the sample polynucleotide is the “sample element” or “sample sequence element”.

A “sample tag” refers to a sequence element used to identify or distinguish different sample polynucleotides, sequence elements or clones present as members of a collection. In general, an individual sample tag is joined to an individual polynucleotide resulting in a collection of “sample-tagged” polynucleotides comprising distinct sample tags. A sample-tagged polynucleotide may comprise one or more distinct sample tags, which are used to distinguish different segments of the polynucleotide. For example, sample tags may be present at the 5′ and 3′ ends of the polynucleotide, or different tags may be distributed at multiple sites in the polynucleotide. The same sample polynucleotide may be associated with more than one sample tag, but to be informative, one sample tag must be associated with only one sample polynucleotide in a collection. It is these informative associations that constitute sample-tagged clones. Methods for designing sample tags are well known in the art as exemplified by, e.g., Brenner (U.S. Pat. No. 5,635,400). In some embodiments of the invention, the sample tags may comprise individual synthetic oligonucleotides each of which has been ligated into a vector, to provide a library or collection of vectors with distinct sample tags or the oligonucleotides are ligated directly to the polynucleotides to be analyzed. In other embodiments, the sample tag may comprise part of the sample sequence element.

“Tagged” as used herein in reference to a polynucleotide means the polynucleotide is derived in one or more steps from a sample-tagged polynucleotide by for example enzymatic, chemical or mechanical means, and the polynucleotide comprises a tag. The “tag” is a sequence element that corresponds to a sample tag and can be used to identify or distinguish the sample tag. Note a sequence element is itself a tag if it is derived from a tag and can be used to identify or distinguish the tag. In many embodiments, the tag and the sample tag are identical. In certain embodiments, the tag comprises the sample tag but contains additional sequence elements. The additional sequence elements may be necessary for example to permit increased hybridization temperatures or to impose structural constraints on the tag. In other embodiments, the sample tag comprises the tag but contains additional sequence elements. For example, two different sample tags that share the same tag may be distinguished by preferential PCR amplification of the tag with primers that are specific to only one tag. Subsequent removal of the priming sequences produces identical tags that can be used to distinguish the different sample tags. During amplification or another step in the invention, the tag could lose all sequence identity with the sample tag. Nevertheless, as long as there exists an identifiable correspondence between the two, information associated with the tag can be related to the sample tag which in turn can be related to the sample polynucleotide. The number of distinct tags required to characterize a collection of sample-tagged polynucleotides will vary. In some embodiments, a one-to-one relationship exists between the tag and the sample tag. In other embodiments, the tags will identify information in addition to the sample identity, for example the terminating nucleotide, the restriction site, etc. Consequently, more distinct tags than distinct sample tags may be used. Finally as outlined above, the same tag may be used to identify more than one sample tag.

A “tag complement” as used herein refers to a molecule that will substantially hybridize to only one tag, or a set of distinguishable tags, among a collection of tags under the appropriate conditions. Different tags that hybridize to the same tag complement may be distinguished for example by different fluorophores, by their ability to hybridize to a second oligonucleotide, etc. Some degree of cross-hybridization by otherwise distinguishable tags can be tolerated, provided the signal arising from hybridization between a tag A and its tag complement A′ is discernable from the cross-hybridization signal arising from hybridization between a different tag B and the tag complement A′. In embodiments where the tag complement is a polynucleotide or sequence element, preferably the tag is perfectly matched to the tag complement. In embodiments where specific hybridization results in a triplex, the tag may be selected to be either double stranded or single stranded. Thus, where triplexes are formed, the term “complement” is meant to encompass either a double stranded complement of a single stranded tag or a single stranded complement of a double stranded tag. Tag complements need not be polynucleotides. For example, RNA and single-stranded DNA are known to adopt sequence dependent conformations and will specifically bind to polypeptides and other molecules (Gold et al., U.S. Pat. No. 5,270,163 & U.S. Pat. No. 5,475,096).

The terms “oligonucleotide” or “polynucleotide” as used herein include linear oligomers of natural or modified monomers or linkages, including deoxyribonucleosides, ribonucleosides, I-anomeric forms thereof, peptide nucleic acids (PNAs), and the like, capable of specifically binding under the appropriate conditions to a target polynucleotide by way of a regular pattern of monomer-to-monomer interactions, such as Watson-Crick type of base pairing, base stacking, Hoogsteen or reverse Hoogsteen types of base pairing, or the like. Usually monomers are linked by phosphodiester bonds or analogs thereof to form “oligonucleotides” ranging in size from a few monomeric units, e.g., 3-4, to several tens of monomeric units, and “polynucleotides” are larger. However the usage of the terms “oligonucleotides” and “polynucleotides” in the art overlaps and varies. The terms are used interchangeably herein. Whenever a polynucleotide is represented by a sequence of letters, such as “ATGCCTG,” it will be understood that the nucleotides are in 5′→3′ order from left to right and that “A” denotes deoxyadenosine, “C” denotes deoxycytidine, “G” denotes deoxyguanosine, and “T” denotes thymidine, unless otherwise noted. Analogs of phosphodiester linkages include phosphorothioate, phosphorodithioate, phosphoranilidate, phosphoramidate, and the like. It is clear to those skilled in the art when polynucleotides having natural or non-natural nucleotides may be employed. Polynucleotides or oligonucleotides can be single-stranded or double-stranded. As used herein, “nucleic acid sequencing reaction” refers to a reaction that carried out on a polynucleotide clone will produce a collection of polynucleotides of differing chain length from which the sequence of the original nucleic acid can be determined. The term encompasses, e.g., methods commonly referred to as “Sanger Sequencing,” which uses dideoxy chain terminators to produce the collection of polynucleotides of differing length and variants such as “Thermal Cycle Sequencing”, “Solid Phase Sequencing,” exonuclease methods, and methods that use chemical cleavage to produce the collection of polynucleotides of differing length, such as Maxam-Gilbert and phosphothioate sequencing. These methods are well known in the art and are described in, e.g., Ausubel, et al., Current Protocols in Molecular Biology, John Wiley, New York, 1997; Gish et al., Science, 240: 1520-1522, 1988; Sorge et al., Proc. Natl. Acad. Sci. USA, 86:9208-12, 1989; Li et al., Nucleic Acids Res., 21:1239-44, 1993; Porter et al., Nucleic Acids Res., 25:1611-7, 1997. The term also includes methods based on termination of RNA polymerase (e.g., Axelrod et al., Nucleic Acids Res., 5:3549-63, 1978).

A “sequencing method” is a broad term that encompasses any reaction carried out on a polynucleotide to determine some sequence from the polynucleotide. The term encompasses nucleic acid sequencing reactions, sequencing by hybridization (Southern, U.S. Pat. No. 5,700,637; Drmanac et al., U.S. Pat. No. 5,202,231;Khrapko et al., U.S. Pat. No. 5,552,270; Fodor et al., U.S. Pat. No. 5,871,928), step-wise sequencing (e.g. Cheeseman, U.S. Pat. No. 5,302,509; Rosenthal, PCT Pat. Pub. No. WO 93/21340; Brenner, U.S. Pat. No. 5,763,175), etc.

A “sequence ladder” refers to a pattern of fragments from one clone resulting from the size separation and visualization of reaction products produced by a “nucleic acid sequencing reaction.” Typically, size separation is accomplished by denaturing gel electrophoresis. The nucleic acid sequence is ascertained by interpreting the “sequence ladder” to determine the identity of the 3 ′ terminal nucleotides of reaction products that differ in length by one nucleotide. Generating and interpreting “sequence ladders” is well within the skill in the art, and is described in, e.g., Ausubel et al., Current Protocols in Molecular Biology, John Wiley, New York, 1997. A “band” in a sequence ladder refers to the clonal population of reaction products that terminate at the same base and so migrate together through the separation medium. A band will have width due to dispersion and diffusion, so it is possible to speak of a part or portion of a band, which means a collection of the clonal population that has migrated more closely together than some other collection.

A “primer” is a molecule that binds to a polynucleotide and enables a polymerase to begin synthesis of the daughter strand. For example, a primer can be a short oligonucleotide, a tRNA (e.g. Panet et al., Proc. Natl. Acad. Sci. USA., 72:2535-9, 1975) or a polypeptide (e.g. Guggenheimer et al., J. Biol. Chem., 259:7807-14, 1984). A “primer binding site” is the sequence element to which the primer binds.

A “sequencing primer” is an oligonucleotide that is hybridized to a polynucleotide clone to prime a nucleic acid sequencing reaction. The sequencing primer is prepared separately, usually on a DNA synthesizer and then combined with the polynucleotide. A “sequencing primer binding site” is the sequence element to which the sequencing primer hybridizes. The sequencing primer binding sites in two different polynucleotides are considered to be the same when the same sequencing primer will efficiently prime the nucleic acid sequencing reaction for both polynucleotides. Of course, mispriming frequently occurs during sequencing reactions, but these artifactual priming sites are minor components of the sequencing reaction products. One skilled in the art will readily understand the difference between mispriming and efficient priming at the sequencing primer binding site.

“Deconvoluting” means separating data derived from a plurality of different polynucleotides into component parts, wherein each component represents data derived from one of the polynucleotides comprising the plurality.

An “array” refers to a solid support that provides a plurality of spatially addressable locations, referred to herein as features, at which molecules may be bound. The number of different kinds of molecules bound at one feature is small relative to the total number of different kinds of molecules in the array. In many embodiments, only one kind of molecule (e.g. oligonucleotide) is bound at each feature. Similarly, “to array” a collection of molecules means to form an array of the molecules.

“Spatially addressable” means that the location of a molecule bound to the array can be recorded and tracked throughout any of the procedures carried out according to the method of the invention.

A “library” refers to a collection of polynucleotides. A particular library might include, for example, clones of all of the DNA sequences expressed in a certain kind of cell, or in a certain organ of the body, or a collection of man-made polynucleotides, or a collection of polynucleotides comprising combinations of naturally-occurring and man-made sequences. Polynucleotides in the library may be spatially separated, for example one clone per well of a microtiter plate, or the library may comprise a pool of polynucleotides or clones. When a reaction is performed on a spatially separated library, the same reaction by definition must be performed separately on every member of the library. When a reaction is performed on a pooled library, the reaction need only be performed once.

“Physical mapping” broadly refers to determining the locations of two or more landmarks in a polynucleotide segment. The term is meant to distinguish genetic mapping methods, which rely on a determination of recombination frequencies to estimate distance between two or more landmarks, from the methods of the present invention, which determine the actual linear distance between landmarks. Similarly, a “physical map” is the product of physical mapping.

“Landmark” broadly refers to any distinguishable feature in a polynucleotide other than an unmodified nucleotide. Landmarks include, by way of example, restriction sites, single nucleotide polymorphisms, short sequence elements recognized by nucleic acid binding molecules, DNase hypersensitive sites, methylation sites, transposon, etc. This definition is meant to distinguish physical mapping from “sequencing”, which refers to determining the linear order of nucleotides in a polynucleotide.

“Fingerprinting” refers to the use of physical mapping data to determine which nucleic acid fragments have a specific sequence (fingerprint) in common and therefore overlap.

“Cloning” as used herein in reference to a polynucleotide refers to any method used to replicate a polynucleotide segment. The term encompasses cloning in vivo, which makes use of a cloning vector to carry inserts of the polynucleotide segment of interest, and what I refer to as cloning in vitro in which one or both strands of a polynucleotide segment of interest is replicated without the use of a vector. Cloning in vitro encompasses, for example, replication of a polynucleotide segment using PCR, linear amplification using a primer that recognizes a portion of the polynucleotide segment in conjunction with an enzyme capable of replicating the polynucleotide, in-vitro transcription, rolling circle replication, etc. Similarly, a “clone” in reference to a polynucleotide means a polynucleotide that has been replicated to produce a population of polynucleotides or sequence elements that share identical or substantially identical sequence. Substantial identity encompasses variations in the sequence of a polynucleotide that sometimes are introduced during PCR or other replication methods. This notion of substantial identity is well understood by those skilled in the art and it applies whenever the identity of polynucleotides is at issue.

“Hybridization” as used herein refers to a sequence dependent binding interaction between at least one strand of a polynucleotide and another molecule. From the context, it is obvious to one skilled in the art whether a double-stranded polynucleotide must be denatured before the binding event. For example, the term includes Watson-Crick type base pairing, Hoogsteen and reverse Hoogsteen bonding, binding of an aptamer to its cognate molecule, etc. “Cross-hybridization” occurs when two distinct polynucleotides can bind to the same molecule or two distinct molecules can bind to the same polynucleotide. In general, cross-hybridization depends on the collection of polynucleotides (or molecules) since two polynucleotides (or molecules) cannot cross-hybridize if they are not in the same collection. Hybridization and cross-hybridization also may be used in reference to sequence elements. For example, two distinct polynucleotides may contain identical sample tags. The polynucleotides cross-hybridize to the tag complement whereas the tags, being identical, do not cross hybridize.

A “common sequence” or “common sequence element” refers to a sequence or sequence element that is or is intended to be present in every member of a collection of polynucleotides.

The term “distinct” as used herein in reference to polynucleotides or sequence elements means that the sequences of the polynucleotides or sequence elements are not identical,

A “pool” is a group of different molecules or objects that is combined together so that they are not isolated from one another and any operation performed on one member of the pool is by necessity performed on many members of the pool. For example, a pool of polynucleotides in solution is simply a plurality of different polynucleotides or clones mixed together in one solution; or each clone may be attached to a solid support, for example an array or a bead, in which case the pool consists of the clones combined together in one solution (e.g. the same fluid container). Similarly, “to pool” means to form a pool.

An “aliquot” is a subdivision of a sample such that the composition of the aliquot is essentially identical to the composition of the sample.

The term “to derive” as used herein in reference to polynucleotides means to generate one polynucleotide from another by any process, for example enzymatic, chemical or mechanical. The generated polynucleotide is “derived” from the other polynucleotide.

The term “amplify” in reference to a polynucleotide means to use any method to produce multiple copies of a polynucleotide segment, called the “amplicon”, by replicating a sequence element from the polynucleotide or by deriving a second polynucleotide from the first polynucleotide and replicating a sequence element from the second polynucleotide. The copies of the amplicon may exist as separate polynucleotides or one polynucleotide may comprise several copies of the amplicon. A polynucleotide may be amplified by, for example a polymerase chain reaction, in vitro transcription, rolling-circle replication, in vivo replication, etc. Frequently, the term “amplify” is used in reference to a sequence element in the amplicon. For example, one may refer to amplifying the tag in a polynucleotide by which is meant amplifying the polynucleotide to produce an amplicon comprising the tag sequence element. The precise usage of amplify is clear from the context to one skilled in the art.

The term “cleave” as used herein in reference to a polynucleotide means to perform a process that produces a smaller fragment of the polynucleotide. If the polynucleotide is double-stranded, only one of the strands may contribute to the smaller fragment. For example, physical shearing, endonucleases, exonucleases, polymerases, recombinases, topoisomerases, etc. will cleave a polynucleotide under the appropriate conditions. A “cleavage reaction” is the process by which a polynucleotide is cleaved.

A “mapping reaction” as used herein refers to any reaction that can be carried out on a polynucleotide clone to generate a physical map or a nucleotide sequence of the clone. Similarly, a “map” is a physical map or a nucleotide sequence.

The term “associating” as used herein in reference to a tagged polynucleotide with a property and a tag complement means determining that the polynucleotide hybridizes to the tag complement. In many embodiments, associating simply means hybridizing a polynucleotide with a known property to a tag complement and detecting the hybridization. In other embodiments, associating means detecting a property of a polynucleotide that is already hybridized to a tag complement. In both cases, the result is information that the polynucleotide has a certain property and in addition hybridizes to the tag complement. The properties of a polynucleotide can include for example the length, terminal base, terminal landmark or other properties according to this invention.

A “junction” as used herein in reference to insertion elements is the DNA that flanks one side of the insertion element.

An “array sequencing reaction” is any method that is used to determine sequences from a plurality of polynucleotides in an array, for example methods described by Brenner (U.S. Pat. No. 5,695,934 and U.S. Pat. No. 5,763,175), Brenner et al. (U.S. Pat. No. 5,714,330), Cheeseman (U.S. Pat. No. 5,302,509), Drmanac et al. (U.S. Pat. No. 5,202,231), Pastinen et al. Genome Res., 7:606-14, 1997, Dubiley et al. Nucleic Acids Res., 25:2259-65, 1997, Graber et al. Genet Anal., 14:215-9, 1999, etc.

A “single-particle detection method” is defined as a method for detecting individual molecules or individual particles where a “particle” is a molecule that is amplified prior to detection in such a way that the amplification products remain associated in a group. Examples of particles include the products of rolling circle amplification (U.S. Pat. No. 5,854,033 and Lizardi, et al., Nat. Genet. 19:225-232, 1998), polonies (Mitra, R. D. et al., Analyt. Biochem. 320:55-65, 2003; Zhang, K. et al., Nature Biotech. 24:680-686, 2006), polynucleotides amplified in-situ on a substrate (e.g. bridge amplification, U.S. Pat. No. 5,641,658), BEAMing (Ghadessy et al. (2001) Proc. Natl. Acad. Sci. USA 98 p 4552; Dressman et al. (2003) Proc. Natl. Acad. Sci. USA 100 p 8817), etc. Single-particle detection methods encompass single-molecule detection methods, such as for example nanopores, atomic force microscopy, scanning tunneling microscopy, scanning electrochemical microscopy, magnetic resonance force microscopy, surface enhanced raman spectroscopy, scanning near-field optical microscopy, etc.

A “Bar code” in reference to a tag or sample-tag comprises a series of sequence elements, referred to as “segments”. These segments are chosen from a set of segments such that different tags comprise different subsets and/or combinations from this set.

### 6.2 Multiplexed Sequencing

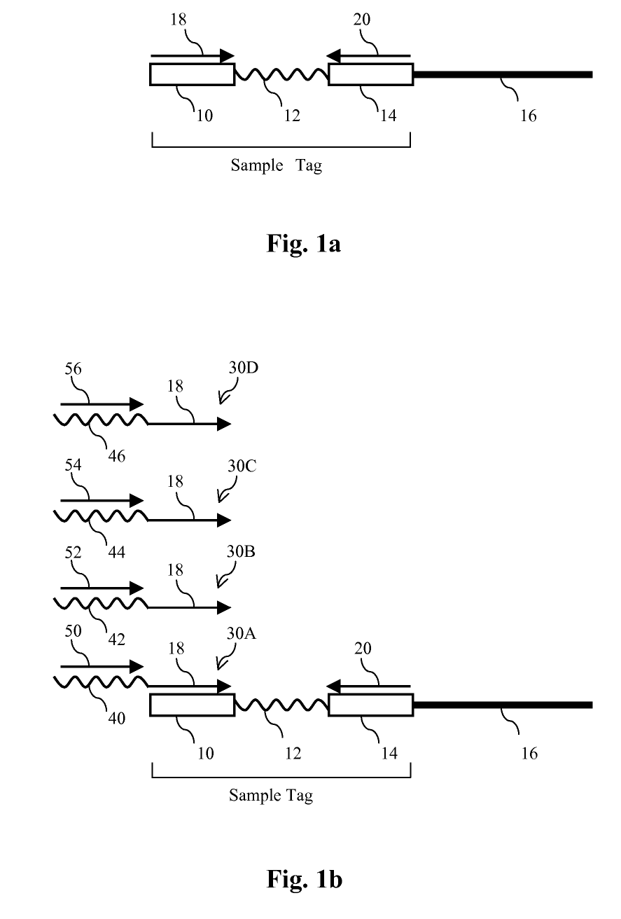

A collection of sample-tagged clones is prepared by joining a set of sample polynucleotides with a set of sample tags so that many of the sample tags (i.e., preferably, at least approximately 35% of the total) are associated with unique sample polynucleotides. A preferred sample tag, as shown in FIG. 1a, comprises a distinct sequence element 12 flanked on both sides by common regions 10 & 14 shared by the other clones. The sample sequence element 16 comprises the sample polynucleotide that is joined to the sample tag. A nucleic acid sequencing reaction is performed on the pooled collection of sample-tagged clones (i.e., Sanger chain-termination method, Maxam & Gilbert chemical cleavage method, etc.) Typically, four separate reactions are performed, which correspond to the four (A, T, G, C) nucleotides. The Sanger method employs the sequencing primer 18, which hybridizes to the sequencing primer binding site in common region 10. In this example, only one sequencing primer binding site is needed for the sequencing reaction to be performed on the pool of sample-tagged clones. Of course, different collections of clones with different common regions comprising different sequencing primer binding sites may be pooled and more than one primer may be utilized, but preferably there will be many more sample-tagged clones than sequencing primer binding sites utilized in the sequencing reaction. One or a limited number of primer binding sites means only a small number of sequencing primers are required for the sequencing reaction, which produces efficient priming and limits spurious priming artifacts.

The products of the sequencing reactions (i.e. the sequence “ladders”) are separated by size and four sets of fractions are collected. Any method of separation may be used that sufficiently resolves the sequencing fragments (i.e. single nucleotide resolution) and permits collection of the fragments in a state compatible with subsequent analysis (i.e. amplification and/or flow cytometry, attachment to a glass slide, etc. see below). Representative methods include polyacrylamide gel electrophoresis, capillary electrophoresis, chromatography, etc. These methods are well known in the art and are described in, e.g. Ausubel et al. Current Protocols in Molecular Biology, John Wiley, New York, 1997; Landers, Handbook of Capillary Electrophoresis, CRC Press, Boca Raton, Fla., 1996; and Thayer, J. R. et al., Methods Enzymol., 271:147-74, 1996. Fractions may be collected, for example, by running the sequencing reactions off the bottom of a gel or column, or each lane of a gel may be sectioned in the direction normal to the direction of electrophoresis (i.e., transversely) and nucleic acid eluted from the sections. Ideally, each fraction corresponds to chain lengths that differ by one nucleotide and any one band is completely contained in only one fraction. Different clones however will display slight variations in band migrations, so fractions may contain only part of a band. The resolution of the sequence ladders will improve with more fractions per band (i.e. smaller fraction size), but the tradeoff is that more work is needed to deconvolute and reconstruct the ladders. Methods for deconvoluting the sequence ladders involve sampling each fraction to determine the relative abundance of any distinct sequence element (12) in the fraction. Deconvolution is discussed in detail below.

A slight variation of the above technique will permit the four sequencing reactions to be run together in one lane. Thus, problems associated with lane-to-lane variation during gel electrophoresis are eliminated. The sequencing primer 18 can tolerate additional sequences at its 5′-end without influencing priming. Consider four different sequences of identical length (40, 42, 44 and 46) added to the 5′-end of primer 18 to make sequencing primers 30A, 30B, 30C and 30D as shown in FIG. 1b. Now, the four chain-terminating reactions are performed with the four different sequencing primers (i.e., ddATP reaction is primed by 30A, ddGTP is primed by 30B, etc.) The four reactions are pooled, separated by size and fractionated. Deconvolution of the fractions includes identification of the distinct sequence element 12 and to which of the four sequence elements (40, 42, 44 or 46) it is joined. Not all sequences of equal length will work equally well for the sequence elements 40, 42, 44 and 46. Preferably, the sequencing primers 30A, 30B, 30C and 30D have minimal secondary structure so their contribution to the mobility of the reaction products during separation is based essentially entirely on length, that is the different primers contribute equally to mobility.

An analogous strategy can be employed to permit pooling of nucleic acid sequencing products generated by the Maxam-Gilbert method (and other chemical cleavage methods). In this case, sequence elements that correspond to 40, 42, 44 and 46 are ligated as adapters to the sample-tagged polynucleotides before or after the reactions, but prior to pooling and separation. That is, the adapter comprising sequence 40 is ligated to polynucleotides subjected to the “A+G” reaction, the adapter comprising sequence 42 is ligated to polynucleotides subjected to the “G” reaction, etc.

Clearly, many different nucleic acid sequencing reactions can be used to practice this invention. The Sanger and Maxam-Gilbert methods are outlined above, but several variations are well known in the art.

Denaturing polyacrylamide gels separate DNA principally by size. There are well known exceptions to this rule (e.g., compressions) that can affect DNA migration. In addition, there is a very slight dependence of mobility on the terminal 3′-base. Therefore, sequencing bands corresponding to equivalent sizes need not be perfectly superimposed. The problem of reconstructing a sequence ladder from the relative levels of any one tag in each fraction becomes a problem of reconstructing a wave from sampled intervals along that wave (picture the readout from an ABI sequencer and this problem becomes clear). Obviously, the more fractions that one collects, the more information one has to reconstruct the parent wave. This is simply a problem in information theory, well known in the art, see for example Stockham et al., U.S. Pat. No. 5,273,632; Allison et al., U.S. Pat. No. 5,748,491; Fujiwara et al., U.S. Pat. No. 4,329,591; Johnson et al., Methods Enzymol., 240:51-68, 1994 and Press et al., in Numerical Recipes in C, Cambridge University Press, pp. 398-470, 1988. By calibrating the fractionation apparatus and/or using internal standards, the appropriate sampling frequency is determined. By optimizing the gel conditions and stressing uniform band intensities and uniform spacing, it may be possible to obtain unambiguous sequence data with fewer fractions than bases. Very few template preparations and sequencing reactions are needed to obtain enormous amounts of sequence information so even elaborate protocols (e.g., cesium banding, formamide gels, etc.) and a variety of nucleotide analogs (e.g. 7-deaza-dGTP and dITP) can be used to produce optimally fractionated sequencing products.

An important aspect of the method, is the ability to construct the clone libraries in a pool of sample tagged vectors (alternatively, the sample tags can be added as adapters and the clones ligated into the same vector, see Sagner et al., U.S. Pat. No. 5,714,318). This approach greatly reduces the effort involved in library construction, but comes at a cost of lost information per pool. For example, consider a library that consists of 500,000 different sample tags. The effort would be enormous to make 500,000 separate libraries and then to pick a single clone from each library. Instead, one library is constructed and about 500,000 transformants are pooled (a very trivial operation). Similarly, the library may be constructed entirely in vitro, and 500,000 clones may be selected by amplifying in vitro a proper dilution of the library so that the amplicons comprise about 500,000 clones. However assuming a normal distribution, only a fraction (1/e=0.37) of the sample tags is expected to be present only once in the library (i.e. attached to only one sample polynucleotide). 37% of the sample tags are expected to be absent from the collection and the remainder will be present two or more times (that is, two or more different polynucleotide clones will contain the same sample tag). Therefore, 63% of the original sample tags will provide no or garbled information. Those original sample tags providing garbled information are readily recognized because more than a single base is identified at each position during the deconvolution step. This loss is well worth the savings in effort. A preferred strategy can increase the information content, such as using 5 million original sample tags and selecting only 500,000 clones, to extract about 90% of the information. Of course, if smaller numbers of clones are to be sequenced, it is feasible to construct separate libraries for each tag and pool one member from each library before performing the sequencing reaction, or even separately perform the sequencing reaction on one member from each library and then pool the reaction products.

### 6.2 Deconvolution

The critical aspect to the instant invention is the means of deconvoluting the fractions of multiplexed sequencing products. Other methods (Strathmann, M. P., U.S. Pat. No. 6,480,791; Wong, W. H., U.S. Pat. No. 5,935,793 & Rabani, E. M., PCT Pat. Pub. No. WO 97/07245) that employ nucleic acid sample tags make use of arrays of tag complements to deconvolute the sequence ladders. The signal from any one feature in the array, which typically comprises one tag complement, represents an average sampling of the fractionated sequencing-reaction products comprising one tag. In other words, the fractionated polynucleotides comprising the same tag are measured simultaneously when a signal is detected from a feature in the array. The strength of this signal depends on the concentration of the tagged polynucleotide and is affected by the overall complexity of tags in the fraction. The instant specification teaches a method whereby the fractions may be deconvoluted by identifying tags with single-particle detection means that need not involve an array of tag complements. In this way, it is possible to circumvent the slow kinetics that result from highly complex hybridization mixtures (i.e. many tags hybridizing to many tag complements). The result can be more rapid and less expensive sequencing.

Considerable effort has gone into the development of methods for sequencing single molecules of DNA (Marziali, A. et al., Annu. Rev. Biomed. Eng. 3:195-223, 2001). To date, no method is capable of consistently resolving individual nucleotides. However, several methods are capable of distinguishing differences between nucleic acids at lower “resolution”. As long as the tags are designed to be distinguishable at this lower resolution, then the method may be applied to “read” the tags and deconvolute the sequence ladders. In fact, any methodology capable of recognizing differences in certain physical characteristics of individual molecules of nucleic acid could potentially be used to practice the instant invention. More generally, a methodology capable of recognizing differences in certain physical characteristics of individual molecules associated with or derived from a nucleic acid may potentially be used to practice the instant invention (for example, fluorophores may be associated with a nucleic acid and a polypeptide may be derived from a nucleic acid). The requirement is the physical characteristics be determined in some way by the tag so that by measuring the physical characteristics the tag is identified.

One method for detecting and characterizing single molecules is to observe changes in ionic current through a nanopore as the molecule of interest traverses or otherwise blocks the nanopore. “Nanopore sequencing” of nucleic acids is an area of intensive research and methods for making and using nanopores are well known in the art (see for example, U.S. Pat. No. 6,428,959; U.S. Pat. No. 5,795,782 and U.S. Pat. No. 6,706,204, which are herein incorporated in their entirety). Though nanopores are not yet capable of discriminating the individual nucleotides in a polynucleotide, they can be used to discriminate between different polynucleotides (see for example, Meller A. et al., Proc. Natl. Acad Sci. USA 97:1079-1084, 2000; Howorka, S. et al., Nat. Biotechnol. 19:636-639; Akeson, M. et al., Biophys. J. 77:3227-3233, 1999; Vercoutere, W. et al., Nat. Biotechnol. 19:248-252)

Discrimination can be improved by including an adapter molecule with the nanopore (see for example, U.S. Pat. No. 6,426,231). As long as the sequence tags comprise distinct sequence elements (12 in FIG. 1) which can be discriminated by the nanopore, then deconvolution of the multiplexed sequencing products is possible.

One method to design sequence tags that can be discriminated by a nanopore takes advantage of the differences in translocation time through a nanopore between single-stranded and double-stranded DNA (see U.S. Pat. No. 6,936,433). Double-stranded DNA must “melt” before it can pass through a nanopore of the appropriate size (e.g. alpha-hemolysin, Sauer-Budge, A. F. et al., Phys. Rev. Lett. 90:238101, 2003; Sutherland, T. C. et al., Biochem. Cell Biol. 82:407-412, 2004). Tags may be designed with regions of double-stranded DNA separated by regions of single-stranded DNA to produce a molecule that may be read like a “bar code”, as taught in U.S. Pat. No. 6,465,193. A series of discernable changes in current through the nanopore will identify the particular bar code in the same way that white and black lines of different sizes are used in the familiar UPC (Universal Product Code) bar-coding system to uniquely identify an item. The double-stranded regions of the tag may be hairpin structures. Tags may also be made double-stranded by, for example, hybridizing oligonucleotides that are complementary to the appropriate regions of the tags. In the latter case, the distinct sequence element (12 in FIG. 1a), would comprise several sub-domains (segments) to which several different oligonucleotides could hybridize. The spacing and order of these sub-domains, which are analogous to the dark lines of a UPC code, determine the identity of the distinct sequence element. The hybridization step would preferably occur after fractionation of the multiplexed sequencing products. In the example of the sequence tag shown in FIG. 1a, it may be preferable to analyze only the tag portion of the sequencing product. The tag may be easily separated from the sequencing product by, for example, hybridizing primer 20 to the sequencing products and synthesizing the complementary strand. It may be advantageous to attach a biotin (or analogous) group to primer 20 so the complementary strand can be purified with for example streptavidin coated beads before interrogation with the nanopore. The oligonucleotides used in the hybridization step could be conjugated to other molecules, for example bulkier groups, that may be more readily discriminated by a nanopore.

The use of single-molecule detection means like nanopores to deconvolute fractionated tagged reaction products means each measurement is interrogating only one tag or tagged molecule at a time. Obviously for a given number of different tags, many times that number of measurements must be made to ensure that all the tags, or almost all the tags, are identified in a fraction. For example, if a fraction contains 100,000 different tags then making 1,000,000 measurements will effectively ensure that all the tags are measured at least once assuming a normal distribution. Because of occasional sequence dependent variations in the separation of DNA molecules by size (compressions, etc.), some fractions may contain parts of two (or more) different bands corresponding to the same tag. For this reason, depending on the accuracy of sequencing that is desired, one may need to increase the number of measurements in the above example to 10,000,000 or more. The exact number of measurements to be performed can be determined empirically as well (e.g. continue measuring until each tag has been read at least 10 times).

Other methods for single-molecule detection include atomic force microscopy, scanning tunneling microscopy, transmission electron microscopy, surface enhanced raman scattering, etc. Though to date no method can consistently discriminate individual bases in a nucleic acid, other small structures can be identified (see for example, Dunlap, D. D. et al., Nature 342:204-206, 1989; Fotiadis, D. et al, Micron 33:385-397, 2002; Seong, G. H. et al. Anal. Chem. 72:1288-1293, 2000; U.S. patent application No. Ser. 10/067,029; U.S. Pat. No. 5,224,376; U.S. Pat. No. 6,002,471). A nucleic acid tag can be designed very easily to encode information in structures larger than individual nucleotides. For example, hairpins of different sizes, spaced along the tag at intervals of different sizes could be used to identify a particular tag just like a traditional bar code described above. Alternately, a single-stranded tag could be hybridized to a partially complementary strand of DNA to produce a series of mismatched base-pairs in the duplex. Again, the size and spacing of mismatches represents the “bar code” that identifies the tag. If larger structures are desired, a protein that binds to mismatched DNA could be incubated with the tag. Other variations to create identifiable domains within a tag are evident to those skilled in the art. In addition, the tags may be amplified to form particles which could provide replicate measurements when using certain single-molecule detection methods such as atomic force microscopy, etc.

Single-molecule or more generally, single-particle detection methods based on fluorescence are amenable to identification of tags. Of course, actual sequence information could be obtained from the tag to identify it, see for example Braslavsky, I et al., Proc. Natl. Acad. Sci. USA 100:3960-3964, 2003; U.S. Pat. No. 6,818,395 and references therein. Alternatively, the sample-tags may be designed to simplify the sequencing reaction. For example, the sequencing instrument sold by 454 Life Sciences, which sequences particles using a sequencing-by-synthesis approach, is capable of distinguishing runs of up to eight nucleotides (homopolymers) or more (Margulies, M. et al., Nature 437:376-380, 2005). Tags designed with homopolymers can be identified with fewer synthesis steps since each step can identify 8 elements (i.e. segments) or more (homopolymers of one base) instead of just one element (a single base). For example, two steps to identify adenine then guanine can identify 64 different tags (AG, AAG, AAAG . . . AGG, AGGG . . . AAAG, AAAGG, AAAGGG . . . etc.).

Information may also be encoded by for example, other bar code approaches for identification with fluorescence-based single particle detection methods. For example, each sample tag may comprise a distinct sequence element (e.g. 12 in FIG. 1a) consisting of twelve 8-base segments for a total length of 96 bases. Each 8-base segment is taken from a set of four unique segments, and the twelve sets of four 8-base segments (12×4=48 8-base segments) are unique. Each tag can be uniquely identified by twelve successive hybridizations of four pooled oligonucleotide probes wherein each oligonucleotide in a pool is labeled with a distinctive fluorophore and is complementary to one segment in a set of four 8-base segments. In other words, a sample tag comprises a distinct sequence element of the following form AₐB_{b}C_{c}. . . KₖLₗ, where a,b,c . . . k,l is 1,2,3 or 4 and each segment A₁, A₂, A₃, A₄, B₁, B₂, . . . through L₄ is a unique 8-base segment. The tag is decoded by first hybridizing a pool of oligonucleotide probes comprising A′₁-l₁, A′₂-l₂, A′₃-l₃ and A′₄-l₄ where A′₁-l₁ is complementary to A₁, A′₂-l₂ is complementary to A₂, etc and lₓ (x=1,2,3,4) are fluorescent labels that are distinguishable. A multiplexed sequencing fraction (i.e. one band in the sequencing ladder) is attached to a substrate, optionally amplified in situ, hybridized with a pool of probes comprising A′ₓ-lₓ (x=1,2,3,4), and images of hybridization to single molecules or single particles are captured as taught by for example U.S. Pat. No. 6,818,395. The labeled probes are stripped by heating or alkali, quenched by over exposure, etc., then the next pool (B′ₓ-lₓ, x=1,2,3,4) is hybridized, imaged and stripped. The process is repeated until all twelve pools of probes have been hybridized and imaged. In this way, 4¹²=16,777,216 different tags can be identified and deconvoluted with twelve hybridizations. Because a single hybridization comprises only 4 labeled oligonucleotide probes, mass action (i.e. high concentrations of probe) will drive the hybridization to completion very quickly (on the order of minutes).

In the above example, all twelve hybridizations may be performed at once if 48 distinguishable fluorescent probes are available. For example, quantum dots may be used. More than one probe could use the same fluorophore if the ratio of fluorophore to probe varies between the probes (e.g. one probe could have two or four flourophores per oligo), see for example Han et al. (2001) Nat. Biotechnol. 19 p 631). In this case, the tags need not be fixed in space, but could be identified for example by flow cytometry (Orden et al. (2000) Anal. Chem. 72 p 37). The tags also may be amplified to form particles, for example by rolling-circle amplification, prior to analysis by flow cytometry. Multiplex analysis of particles by flow cytometry using different ratios of fluorophores is taught by Chandler et al. in U.S. Pat. No. 5,981,180.

Numerous methods, in addition to fluorescence, have been developed for encoding and decoding beads for combinatorial chemistry applications. These methods are readily adapted to decoding/deconvoluting for example barcode-type nucleic acid tags by flow cytometry, fixed-position analysis, etc. as described above for fluorophores. For example, different Raman tags may be associated with each complementary oligo (A′ₓ-rₓ, . . . L′ₓ-rₓ, where x represents the Raman tag, see e.g. Mulvaney et al. (2003) Langmuir 19 p 4784) plasmon-resonant particles (e.g. Schultz et al. (2000) Proc. Natl. Acad. Sci. USA 97 p 996), nanobarcode particles (e.g. Nicewarner-Pena, et al. Science 294 p 137, 2001), etc. To summarize, the entire sequencing process with barcode type tags as described above may be performed as follows: (1) sample tags are joined to templates en masse as described in U.S. Pat. No. 6,480,791 (2) A sequencing reaction is performed on the pool of sample-tagged templates and the reaction products are fractionated as described in U.S. Pat. No. 6,480,791; (3) Each fraction is fixed to a surface (e.g. glass slide), optionally amplified and sequentially hybridized to the pools of labeled complementary oligos (1^{st} pool=A′₁-l₁, A′₂-l₂, A′₃-l₃ and A′₄-l₄), imaged and stripped. Alternatively, distinct labels may be attached to each complimentary oligo and all the oligos are hybridized at once then imaged. In this latter case of distinct labels, the fraction need not be fixed to a substrate. Rather it could be optionally amplified (e.g. rolling circle amplification which produces many replicates of the tag that remain associated, see e.g. Lizardi et. al. (1998) Nat. Genet. 19 p 225), hybridized to the complementary oligos, optionally separated from unhybridized oligos (for example by size) and examined by flow cytometry or some equivalent means (e.g. Raman flow cytometry). Note the complementary oligos may be peptide nucleic acids or any other molecule that specifically interacts with a segment of the tag. A slight modification to the procedure would allow the use of microemulsion amplification (Ghadessy et al. (2001) Proc. Natl. Acad. Sci. USA 98 p 4552; Dressman et al. Proc. Natl. Acad. Sci. USA 100:8817-8822, 2003) prior to flow cytometry or fixed-position imaging. If the microemulsions are not broken, then the complementary oligos could be present prior to amplification in a quenched form for example Molecular Beacons (Tyagi et al. (1998) Nat. Biotechnol. 16 p 49).

The deconvolution of tags by single-particle detection methods has applications beyond sequencing. Methods for physical mapping and determining the locations in the genome of insertion elements as taught in U.S. Pat. No. 6,480,791 rely on the deconvolution of tagged polynucleotides with microarrays. The deconvolution of the tagged reaction products may equally be achieved as described in the instant specification.

### 7. EXAMPLES

### 7.1 Example 1

In this example, sequence information is obtained from about 1,500 polynucleotides. Sample tags are designed as shown in FIG. 1a, where a distinct sequence element 12 is flanked by two common sequence elements 10 and 14. Sequence element 12 comprises six variable sub-elements (or segments): A, B, C, D, E and F where each sub-element is selected in order from a set of four sequence elements that are 10 bases in length (A₁ to A₄, B₁ to B₄, etc.) and a common element G. There are 4⁶=4096 possible sequence tags. The sample tags comprise the sequence elements in the following order: 10-Aₘ-Bₙ-Cₒ-Dₚ-E_{q}-Fᵣ-G-14 where m,n,o,p,q,r=1,2,3 or 4. Each of the 6×4=24 sub-elements is designed to have the same melting temperature.

The 4096 sample tags are synthesized by standard means on an oligonucleotide synthesizer using a split and pool approach. Specifically, sequence element G-14 is first synthesized on standard CPG (controlled pore glass) beads. These beads are divided into four aliquots. F₁ is synthesized on one aliquot, F₂ on another, F₃ on the third and F₄ on the fourth aliquot. The beads are pooled again and divided into four aliquots on which are synthesized E₁ through E₄. The beads are pooled and the process is repeated with the remaining 16 sequence elements. The beads are pooled and sequence element 10 is synthesized on all the beads.

The oligonucleotides are cloned into a plasmid vector by standard means to make a pool of sample-tagged vectors. Mouse genomic DNA is ligated to the pooled vectors at a restriction site designed to be just downstream of sequence element 14 and transformed into E. coli using standard protocols (see for example U.S. Pat. No. 6,480,791 Example 2). About 4000 transformants are selected at random and pooled in preparation for sequencing.

The pool of sample-tagged polynucleotides is sequenced by standard Sanger chain termination protocols. The products are separated by size on a polyacrylamide gel and sequencing bands are excised from the gel and eluted as described in U.S. Pat. No. 6,480,791 Example 1.

Each band is amplified with primers 18 and 20 (FIG. 1a) using the microemulsion “BEAMing” protocol taught by Dressman (Proc. Natl. Acad. Sci. USA 100:8817-8822, 2003; Nat. Methods 3:551-559, 2006). Primer 20 is attached to paramagnetic beads about 1 um in diameter (Dynal Biotech) coated with streptavidin. The result is a collection of beads for each band wherein a single bead comprises a clonal population of tags (i.e. a “particle”) and the tags are in single-stranded form.

Each collection of beads is hybridized with a pool of 25 oligos. 24 oligonucleotides each comprise the sequence of one of the sub-elements and the 25^{th} oligo (oligo G) comprises the sequence element G. The 24 oligos are of the form Zₓ-L(Z)ₓ, where Z is sequence element A, B, C, D, E or F and L(Z) is a fluorescent label that differs between the 6 sets of sub-elements but is the same for all members of a set. Within a set, the number of labels attached to each oligonucleotide varies from one to four. The labels are fluorescein (FITC)=L(A), phycoerythrin (PE)=L(B), Peridinin chlorophyll protein (PerCP)=L(C), PE-Cy7=L(D), allophycocyanin (APC)=L(E) and APC-Cy7=L(F). The set of 24 oligos is A₁ coupled to one FITC label, A₂ coupled to two FITC labels, A₃ coupled to 3 FITC labels, A₄ coupled to four FITC labels, B₁ coupled to one PE label, B₂ coupled to 2 PE labels and so on. Oligo G is labeled with one copy of APC-Cy5.5.

After hybridization to the pool of 24 oligonucleotides and washing, the collection of beads is analyzed by flow cytometry using the FACSCanto system (BD Biosciences). Each bead shows variations in signals from the six labels relative to the baseline label provided by oligo G. The variation in signal for each label is determined by the different number of labels attached to each oligonucleotide in the pool of 24 oligonucleotides. Consequently, the relative intensities identify the sequence elements which in turn identifies the specific tag associated with a bead. In this way, the tags in each band are determined and the sequence ladder for each sample-tagged polynucleotide is deconvoluted. Only about 1500 of the 4000 sample-tagged clones are sequenced in this example due to a normal distribution of the 4096 sample-tags among the clones. The remaining clones due not have unique sample-tags in this population.

### 7.2 Example 2

This example demonstrates deconvolution by rolling-circle amplification and flow cytometry. Sample-tagged polynucleotides are prepared, sequenced, separated and sequencing bands are eluted as described above in Example 1. The tagged polynucleotides in a band are then amplified by using a padlock probe and rolling-circle amplification as taught by Lizardi (U.S. Pat. No. 5,854,033 and Nat. Genet. 19:225-232, 1998). An open circle probe is synthesized by joining together primers 18 and 20 (FIG. 1a) with an intervening sequence element of 50 nucleotides. The open circle probe is converted to a padlock by extending the probe through the tag followed by ligation. Hyperbranched rolling-circle amplification (HRCA) of the padlock is initiated with primers 18 and 20. The amplified tags are hybridized to the pool of 25 oligonucleotides described above in Example 1 and the hyperbranched products are condensed as described by Lizardi (ibid). These condensed particles are analyzed by flow cytometry as described above.

### 7.3 Example 3

This example demonstrates deconvolution by bridge amplification and fluorescent imaging. Sample-tagged polynucleotides are prepared, sequenced, separated and sequencing bands are eluted as described above in Example 1. The tagged polynucleotides in a band are immobilized on a glass slide and subjected to bridge amplification with primers 18 and 20 as described in U.S. Pat. No. 5,641,658 and U.S. Pat. No. 7,115,400. A series of 6 hybridizations is used to identify the tags in a band. Each hybridization includes 4 labeled oligonucleotides corresponding to the four sub-elements in A, B, C, D, E and F described above. In group A, A₁ is labeled with 6-FAM, A₂ with NED, A₃ with HEX and A₄ with ROC (dyes sold by Applied Biosystems, Inc.). The slide is imaged by total internal reflectance microscopy as described (Braslavsky, I et al., Proc. Natl. Acad. Sci. USA 100:3960-3964, 2003; U.S. Pat. No. 6,818,395). Oligonucleotides are removed from the slide by rinsing in 0.1M NaOH and the second group of oligonucleotides (B1 to B4 labeled with the same 4 dyes as above) are hybridized and imaged. This process is repeated 4 more times with the remaining sets of oligonucleotides. Each tag is identified by the pattern of dyes that hybridize to the same location on the slide.

### 7.4 Example 4

In this example, restriction maps are obtained for about 1500 polynucleotides. About 4,000 sample-tagged clones are pooled as described in Example 1. The clones are subjected to partial restriction digestion, separated by agarose electrophoresis and transferred to blotting paper as described in U.S. Pat. No. 6,480,791 (see example 3). The blotting paper is sectioned and the partial digestion products are eluted. The tags in each eluted fraction are then identified by the method described above in Example 2. The partial restriction map for each sample-tagged clone is reassembled from the fractions that contain the corresponding tag.

### 8.INCOPORATION BY REFERENCE

The contents of all cited references (including literature references, patents, and patent applications) that may be cited throughout this application are hereby expressly incorporated by reference.

### 9.EQUIVALENTS

The invention may be embodied in other specific forms without departing from the spirit or essential characteristics thereof The foregoing embodiments are therefore to be considered in all respects illustrative rather than limiting of the invention described herein. Scope of the invention is thus indicated by the appended claims rather than by the foregoing description, and all changes that come within the meaning and range of equivalency of the claims are therefore intended to be embraced herein.

I claim:

1. A method for analyzing polynucleotides, comprising:
a) preparing a collection of sample-tagged clones from the polynucleotides,
b) performing a reaction on the sample-tagged clones to generate a plurality of tagged reaction products of different sizes,
c) separating the reaction products by size,
d) collecting fractions of the separated reaction products,
e) deconvoluting the reaction products by using a single-particle detection method to identify the tags in the fractions.

2. The method of claim 1, wherein the tags comprise bar codes.

3. The method of claim 2, wherein the bar codes comprise segments of 2 nucleotides or more.

4. The method of claim 2, wherein the single-particle detection method comprises hybridizing oligonucleotides that are complementary to segments of the bar codes.

5. The method of claim 1, wherein the separated reaction products are amplified to produce particles comprising tags.

6. The method of claim 1, wherein the reaction is a sequencing reaction.

7. The method of claim 1, wherein the reaction is a cleavage reaction.

8. The method of claim 1, wherein the polynucleotides comprise junctions from insertion elements.

9. The method of claim 1, wherein the single-particle detection method comprises the use of nanopores.

10. The method of claim 1, wherein the single-particle detection method comprises the use of flow cytometry.

11. The method of claim 1, wherein the single-particle detection method comprises the use of microscopy.

12. The method of claim 1, wherein the single-particle detection method comprises a single-molecule detection method.
